(19) United States
(12) Patent Application Publication
(10) Pub. No.: US 2016/0235635 A1
(43) Pub. Date: Aug. 18, 2016

(54) FORMULATIONS INCLUDING SILVER NANOPARTICLES AND METHODS OF USING THE SAME

(71) Applicant: UNIVERSITY OF SOUTH ALABAMA, Mobile, AL (US)

(72) Inventors: Ajay Pratap Singh, Mobile, AL (US); Sumit Arora, Mobile, AL (US); Seema Singh, Mobile, AL (US)

(73) Assignee: University of South Alabama, Mobile, AL (US)

(21) Appl. No.: 15/029,161

(22) PCT Filed: Oct. 16, 2014
(86) PCT No.: PCT/US2014/060939

§ 371 Date: Apr. 13, 2016

Related U.S. Application Data
(60) Provisional application No. 61/961,504, filed on Oct. 16, 2013.

(51) Int. Cl. A61K 8/19; G01N 33/53; A61K 9/00; A61K 9/16; A61Q 17/04; A61K 33/38

(52) U.S. Cl. CPC A61K 8/19; A61Q 17/04; A61K 33/38; A61K 9/0014; A61K 9/16; G01N 33/5308; A61K 2800/596; A61K 2800/413; G01N 2800/20

(57) ABSTRACT

A method for reducing or inhibiting damage to skin is disclosed herein. In some embodiments, the damage is ultraviolet (UV) radiation-induced damage. In some embodiments, the method comprises applying to the skin prior to exposure to radiation damaging source, a formulation containing an effective amount of silver nanoparticles (AgNPs) A method for treating damaged skin, and formulations include AgNPs are also disclosed.

### CROSS-REFERENCE TO RELATED APPLICATIONS

The present application claims the benefit of the filing date of U.S. Provisional Patent Application No. 61/961,504, filed Oct. 16, 2013, the disclosure of which is hereby incorporated herein by reference.

### FIELD OF THE INVENTION

The present invention relates to formulations including silver nanoparticles and methods of treatment including the same.

### BACKGROUND OF THE INVENTION

Over-exposure to UV light is known to cause damage to skin and overall health. For example, the incidence of skin cancer has been increasing at an alarming rate over the past few years in the United States. Each year, there are more new cases of skin cancer than the combined incidence of cancers of the breast, prostate, lung and colon. According to reports (Lazovich et al., Cancer Epidemiol Biomarkers Prev. 2012; 21:1893-1901; Lomas et al., Br J Dermatol. 2012; 166:1069-1080; Siegel et al., CA Cancer J Clin. 2014; 64:9-29). Skin cancer constitutes nearly 30% of all newly diagnosed cancer cases worldwide. Solar ultraviolet (UV) radiation, particularly its UVB component (290-320 nm), is an established cause of about 90% of skin cancers. The incidence, morbidity and mortality rates of skin cancers are increasing and, therefore, continue to pose a significant public health concern.

UV exposure can induce skin cancer due to its ability to damage skin cells at various levels. More specifically, it is believed that UV radiation damages skin cells by forming dimers in DNA between adjacent pyrimidine residues, potentially leading to UV “signature” mutations that can accumulate over time.

UV-induced irradiation damage to skin cells, for example, to the DNA of skin cells can be caused by formation of dimeric photoproducts, e.g., DNA lesions, between adjacent pyrimidine bases on the same DNA strand. For example, such dimeric photoproducts can include cyclobutane pyrimidine dimers (CPDs) and (6-4)-dipyrimidine photoproducts. Among mechanisms in human cells to avoid potential mutation in UV-induced-damaged DNA is to repair the damage by nucleotide excision repair (NER) before replication.

At least 50% of UV-induced irradiation damage to skin cells has been attributed to the formation of reactive oxygen species (ROS). Exemplary ROS can include superoxide radical (O2.—), hydrogen peroxide (H₂O₂), and hydroxyl radical (.OH), among others. For example, ROS can cause strand breaks in DNA and base modifications including oxidation of guanine residues to 8-hydroxydeoxyguanosine (8-OHdG), an oxidized nucleoside of DNA, which plays a crucial role in mutagenesis. 8-OHdG is a miscoding lesion causing G to T transversion and is shown to be a ubiquitous maker of oxidative stress. Base change, such as 8-OHdG, can be repaired by base excision repair (BER) system using glycosylase in combination with replication protein A (RPA), proliferating cell nuclear antigen (PCNA) and AP endonuclease.

Exposure to UV radiation, such as UVB radiation (280-320 nm), is also known to cause apoptosis or cell death, which causes the formation of sunburn cells. Pro-apoptotic (e.g., Bad and Bax) and anti-apoptotic proteins (e.g., Bcl-2 and Bcl-xL), control the process of apoptosis through release/activation of caspases and PARP.

DNA lesions activate checkpoint pathways that regulate specific DNA-repair mechanisms in the different phases of the cell cycle. Checkpoint-arrested cells resume cell-cycle progression once damage has been repaired, whereas cells with unrepairable DNA lesions undergo permanent cell-cycle arrest and subsequent apoptosis. For example, NER and BER pathways are active in G1 phase and repair CPDs and oxo-G, respectively. However, in some instances, a cell may enter into the cell cycle without getting its DNA repaired, which over the course of several cell cycles may lead to accumulation of carcinogenic gene mutations and thus cellular transformation.

Therefore, mitigation of UV-induced DNA damage has been highly desired. For example, topical sunscreen formulations have been used for protection against UV radiation-induced skin injury and carcinogenesis. These formulations include substances that reflect, scatter or absorb UV radiation and thus limit its exposure to the skin cells. Even though these sunscreen formulations have high sun protection factor (SPF) potential, they have been unable to contribute significantly in reducing the incidence of UV-induced skin cancer. (Krause et al., Int J Androl. 2012;35:424-436). Further, studies have shown that the main components of these sunscreen formulations, such as titanium oxide (TiO₂) and zinc oxide (ZnO), can have inflammatory or toxic effects on normal skin cells. (Ghosh et al., J Appl Toxicol. 2013;33:1097-1110; Newman et al., J Am Acad Dermatol. 2009; 61:685-692; Yu et al., Toxicol In Vitro. 2013;27:1187-1195)

Therefore, there is a continued need in the art for safe and effective formulations that reduce UV exposure to normal cells without undesired side effects, and/or repair UV exposure-induced damage.

### SUMMARY OF THE INVENTION

A first aspect of the present invention is directed to a method for reducing or inhibiting damage to skin that entails applying to the skin prior to exposure to a damaging source, a formulation containing an effective amount of silver nanoparticles (AgNPs). In some embodiments, the damaging source is ultraviolet (UV) radiation.

A second aspect of the present invention is directed to a method for treating skin damage that entails applying to the damaged skin a formulation containing an effective amount of silver nanoparticles.

In some embodiments of the methods, the UV radiation is radiated from the sun, and the composition is used as a sunscreen or sunblock. In other embodiments, the UV radiation is from a synthetic radiation source.

A further aspect of the present invention is directed to a formulation containing an effective amount of the silver nanoparticles and at least one dermatologically or cosmetically acceptable carrier. The silver nanoparticles may be formulated into a variety of formulation types for purposes of application to the skin.

In some embodiments, the formulation may be a sunscreen, and further include at least one UV absorbing agent.

Without intending to be bound by any particular theory of operation, Applicant believes that the silver nanoparticles reduce or inhibit several cell functions that may occur as a result of exposure to a damaging source, such as from UV light or another damaging source, including cell apoptosis, replication of mutated cells, ROS generation, and induction of G1/S phase cell cycle arrest, and may also repair DNA lesions, such as cyclobutane pyrimidine dimers (CPDs).

### DESCRIPTION OF THE DRAWINGS

FIG. 1A depicts an electron micrograph of chemically synthesized silver nanoparticles (AgNPs),

FIG. 1B depicts particle size distribution of AgNPs.

FIG. 2 depicts a stability analysis of AgNPs.

FIGS. 3A-D depict characteristic surface plasmon peak for silver nanoparticles (AgNPs) after (A) synthesis, (B) two, (C) four, and (D) six months.

FIG. 4 depicts cytotoxicity of AgNPs after 12, 24 and 48 hours.

FIGS. 5A-B depict morphological changes and percent of surviving HaCaT cells after treatment with AgNPs prior to UVB-exposure.

FIGS. 6A-E depict percent apoptosis in HaCaT cells after treatment with AgNPs prior to UVB-exposure.

FIG. 7 depicts cyclobutane pyrimidine dimers (CPDs) content in HaCaT cells of FIG. 6.

FIGS. 8A-E depict distribution of cells in different cell cycle stages G1, S, and G2 in HaCaT cells of FIG. 6.

FIGS. 9A-B depict expression of cell cycle and survival-related proteins in HaCaT cells of FIG. 6.

FIG. 10 depicts levels of nucleotide excision repair (NER) genes in HaCaT cells of FIG. 6.

FIGS. 11A-E depict reactive ion species (ROS) content in HaCaT cells of FIG. 6.

FIG. 12 depicts cyclobutane pyrimidine dimers (CPDs) content in HaCaT cells after pretreatment with AgNPs prior to UVB-exposure.

FIG. 13 depicts cyclobutane pyrimidine dimers (CPDs) content in UV-exposed HaCaT cells after post treatment with AgNPs.

### DETAILED DESCRIPTION

Formulations including silver nanoparticles (AgNPs) and methods of treatment including the same are disclosed herein. In some embodiments, formulations and methods of treatment using the same may advantageously protect skin cell from UV induced damage and/or repair skin cells after UV induced damage. Further embodiments of the present invention are disclosed herein.

### Silver Nanoparticles (AgNPs)

The term “silver nanoparticles” as used herein refers to particles including silver (Ag) that have at least one dimension less than about 100 nanometers (nm) in length and does not apply to particles under 100 nm that occur naturally or are by-products of other processes such as welding fumes, fire smoke, or carbon black. In some embodiments, the silver nanaparticles can be spherical. The term “particle size” as used herein refers to the length of at least one dimension of a nanoparticle. The particle size typically ranges in size from less than about 100 nm, from less than about 50 nm, or from about 1 to about 50 nanometers (nm). In some embodiments, greater than about 75% of the particles may have a particle size ranging from about 10 to about 40 nm with an average particle size of about 25 nm.

The AgNPs can be included in the formulation in amounts ranging from about 0.002% to about 3 weight percent (wt %), based on the total weight of the composition. In some embodiments, the amounts may range from about 0.002% to about 2 wt %, and in yet other embodiments from about 0.002 wt % to about 1.0 wt %.

The AgNPs suitable for use in the present invention and methods for their preparation are known in the art. For example, one synthesis method includes a reduction reaction using a silver-containing salt in the presence of a reducing agent and a colloidal stabilizing agent, as discussed below with regards to Example 1. Other synthesis methods include top down techniques through breakage of silver metal by laser ablation and beam electron radiation. Thermal decomposition of silver bulk followed by vapor condensation is another top-down menthod for silver nanoparticles synthesis. (See, e.g., Silvert et al., J Mater Chem. 1997; 7(2):293-299; Tsuji et al., Navaladian et al; Nanoscale Res Lett. 2007;2:44-48; Appl Surf Sci. 2002;202:80-85; Li and Zhang; J Nanopart Res. 2010; 12:1423-1428).

### Formulations

The AgNPs can be formulated in a variety of compositions for purposes of topical application. Such formulations or compositions include sunscreens, sunburn relief formulations, hand, body and/or facial moisturizers, topical analgesics, and topical skin treatments, such as anti-acne or anti-fungal medications, as are known in the field of skin care. Representative formulations include lotions, sprays (aerosols), creams, ointments, milks, foams, mousses, tonics, gels, and sticks.

The formulations include at least one additional dermatologically or cosmetically acceptable ingredient, such as a carrier. As used herein the term “carrier” will be understood by one of ordinary skill in the art to mean any component that can be used as a delivery vehicle to facilitate application and prolonged contact of the AgNPs to the skin, and which are inert and non-toxic to and compatible and with the skin. Thus, depending upon the nature of the formulation, acceptable carriers include solids, semi-solids and liquids. If a liquid carrier is used, the formulation is in the form of a dispersion, e.g., AgNPs in suspension in the liquid carrier.

Carriers for topical application of active agents are known in the determatological and cosmetic arts. Representative examples include aqueous media or liquids including water, and non-aqueous media or liquids including methylene chloride, chloroform, aliphatic and aromatic chlorinated liquids , alcohols (e.g., lower (e.g., C2-C6) alkanols such as ethanol and lower (e.g., C2-C6) glycols and polyols), diethyl ether, and lower alkyl esters such as ethyl acetate. Oils can include mineral oils, such as paraffinic oils, naphthenic oils, and aromatic oils, olive oil, plant oils, animal oils or other oils. Exemplary mineral oils may include cyclohexane. Polymers include homopolymers, copolymers, synthetic or naturally derived polymers of acrylamide and its derivatives, methacrylamide and its derivatives, polyamides, polyurethanes, polymers having no particular backbone but with urethane segments or tertiary amine groups in the side chains, other polymers predominantly polar in nature or co-polymers having a portion that is derived from polar co-monomers, methaacrylamide, substituted acrylamides, substituted methaacrylamides, acrylic acid, methacrylic acid, hydroxyethyl methacrylate, acrylonitrile, 2-acrylamido-2-methylpropane sulfonic acid and its salts (sodium, potassium, ammonium), 2-vinyl pyrrolidone, 2-vinyl oxazoline, vinyl acetate, maleic anhydride. The amount of the carrier in in the formulations generally ranges from about 90 to about 99.98 wt %.

In some embodiments, the carrier includes both an aqueous and a non-aqueous liquid and is in the form of an emulsion. Exemplary emulsions can include oil-in-water, water-in-oil, water-in-oil-in-water, and oil-in-water-in-silicone, among others. A non-aqueous or oil phase of the emulsion may include petrolatum, mineral oils, triglycerides of capric or caprylic acid, furthermore natural oils, such as, for example, castor oil; plant oils such as olive oil, sunflower oil, soya oil, peanut oil, rapeseed oil, almond oil, palm oil, coconut oil, palm kernel oil; fats, waxes and other natural and synthetic fatty substances, such as esters of fatty acids with alcohols having a low carbon number, for example with isopropanol, propylene glycol or glycerol, or esters of fatty alcohols with alkanoic acids having a low carbon number or with fatty acids; and silicone oils, such as polysiloxanes (e.g., dimethylpolysiloxanes, diethylpolysiloxanes, and diphenylpolysiloxanes).

By way of additional examples, the oil phase of the emulsion may include ingredients such as esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or esters of aromatic carboxylic acids and saturated and/or unsaturated, branched and/or unbranched alcohols having a chain length of 3 to 30 C atoms. Ester oils of this type may include isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic semi-synthetic and natural mixtures of esters of this type, for example jojoba oil.

By way of further example, the oil phase may include branched and unbranched hydrocarbons and waxes, silicone oils, dialkyl ethers, fatty alcohols, and fatty acid triglycerides, specifically the triglycerol esters of saturated and/or unsaturated, branched and/or unbranched alkanecarboxylic acids having a chain length of 8 to 24 C atoms, in particular 12-18 C atoms. The fatty acid triglycerides may include synthetic, semi-synthetic and natural oils.

The emulsion can include emulsifiers or surfactants. Broadly, surfactants may be anionic, non-ionic or amphoteric. In some embodiments, an O/W emulsion may include an emulsifier such as polyglyceryl-2 dipolyhydroxystearate, glyceryl stearate citrate, glyceryl stearate, polyglyceryl-3 methylglucose distearate, stearic acid, PEG-40 stearate, sodium cetearyl sulfate, hydrogenated cocoglycerides, and one or more coemulsifiers, such as, for example, fatty alcohols, in particular cetearyl alcohol and/or stearyl alcohol, and wherein the oil phase may include oil components such as butylene glycol dicaprylate/dicaprate, dicaprylyl ether, C12-15-alkyl benzoates, C18-38-fatty acid triglycerides, dibutyl adipate and cyclomethicone.

In some embodiments, a W/O emulsion may include an emulsifier such as polyglyceryl-2 dipolyhydroxystearate and PEG-30 dipolyhydroxystearate, and wherein the oil phase includes oil components such as butylene glycol dicaprylate/dicaprate, liquid paraffin, C12-15-alkyl benzoates, C18-38-fatty acid triglycerides, isopropyl stearate and cetyldimethicone.

In embodiments wherein the oil phase includes a silicone oil, a silicone emulsifier can be used. Representative examples include alkylmethicone copolyols and/or alkyldimethicone copolyols (e.g., dimethicone copolyols, cetyldimethicone copolyol, cyclomethiconedimethicone copolyol, laurylmethicone copolyol, and octyldimethicone ethoxyglucoside.

In some embodiments, the emulsion is in the form of a sprayable emulsion, which are known in the art, and include an emulsifier having a lipophilicity that is temperature-dependent (i.e., lipophilicity increases by increasing the temperature and decreases by lowering the temperature), examples of which include polyethoxylated fatty acids (PEG-100 stearate, PEG-20 stearate, PEG-150 laureth, PEG-8 distearate) and polyethoxylated fatty alcohols (ceteareth-12, ceteareth-20, isoceteth-20, beheneth-20, laureth-9 etc.) and alkyl polyglycosides (cetearyl glycoside, stearyl glycoside, palmityl glycoside etc.).

In some embodiments, the AgNPs are formulated in a carrier that includes water and a water miscible liquid such as a lower alcohol or a lower polyol. For example, aqueous-alcoholic mixtures (e.g., water/ethanol) may include from greater than 0% by weight to 90% by weight of ethanol.

In some embodiments, the composition is in the form of an aqueous gel. As used herein the term “gel” refers to a nonfluid colloidal network or polymer network that is expanded throughout its volume by a fluid. The fluid can be aqueous or non-aqueous media, such as water, alcohol, or organic solvents including those disclosed herein, among others. Exemplary aqueous gelling agents include acacia, alginic acid, bentonite, Carbopols® (also known as carbomers or cross-linked polyacrylates), carboxymethylcellulose, ethylcellulose, gelatin, hydroxyethylcellulose, hydroxypropyl cellulose, magnesium aluminum silicate (Veegum®), methylcellulose, poloxamers (Pluronics®), polyvinyl alcohol, sodium alginate, tragacanth, xanthan gum, copolymers of C10-30-alkyl acrylates and one or more monomers of acrylic acid, of methacrylic acid or esters thereof. The INCI name of such compounds is “Acrylates/C 10-30 Alkyl Acrylate Crosspolymer”. The Pemulen® grades TR 1, TR 2 and TRZ from Goodrich (Noveon) are one such commercially available product.

In other embodiments, the composition is in the form of a non-aqueous gel and further contains a lipophilic gelling agent.

The composition can be in the form of an aerosol or non-aerosol. As used herein the term “aerosol” refers to a suspension of fine particles (e.g., AgNPs) in a propellant gas. Exemplary propellant gases can include trichhlorofluoromethane, dichlorodifluoromethane, difluoroethane, dimethylether, propane, n-butane or isobutane, among others. The aerosol is typically packaged under pressure in a container, where a release valve on the container is used to emit the pressurized suspension in the form of a mist propelled the propellant gas. As used herein the term “non-aerosol” refers to the suspension of fine particles (e.g., AgNPs) in a liquid, such as water or another liquid capable of stably suspending fine particles. The liquid can include a buffer, such as citrate or other buffering agents. The non-aerosol is typically packaged in a container having an atomizer attached thereto. Exemplary atomizers can include pump-sprayers. The atomizer causes the non-aerosol to mix with an amount of air which can then be emitted as small droplets.

In another embodiment, the composition is in the form of a water/alcohol mixture, wherein the alcohol (e.g., lower alkanol or polyol is present in an amount. greater than 0 to about 90% of the composition.

### Additional Components

The formulations may further include one or more auxiliary ingredients including, for example, absorbents, abrasives, anti-acne agents, anticaking agents, antifoaming agents, antimicrobial agents, antioxidants, binders, biological additives, buffering agents, bulking agents, chelating agents, chemical additives, colorants, cosmetic astringents, cosmetic biocides, denaturants, drug astringents, external analgesics, structuring agents, film formers, fragrance components, humectants or moisturizers, opacifying agents, pH adjusters, plasticizers, preservatives, propellants, reducing agents, skin protectants, solvents, suspending agents (nonsurfactant), ultraviolet light absorbers, viscosity increasing agents (aqueous and nonaqueous), gums, thickening agents, such as silica, polysaccharides, and others known in the art.

The formulation can include humectants or moisturizers. Exemplary humectants or moisturizers include guanidine; glycolic acid and glycolate salts (e.g., ammonium and quaternary alkyl ammonium); lactic acid and lactate salts (e.g., ammonium and quaternary alkyl ammonium); aloe vera in any of its variety of forms (e.g., aloe vera gel); polyhydroxy alcohols such as sorbitol, glycerol, hexanetriol, propylene glycol, butylene glycol, hexylene glycol and the like; polyethylene glycols; sugars and starches; sugar and starch derivatives (e.g., alkoxylated glucose); hyaluronic acid; lactamide monoethanolamine; acetamide monoethanolamine. The formulation can include humectants or moisturizers in amounts generally ranging from about 10 to about 30 wt %.

The formulation can include gums. Exemplary gums can include, but are not limited to, acacia, agar, algin, alginic acid, ammonium alginate, amylopectin, bentonite, calcium alginate, calcium carrageenan, carnitine, carrageenan, corn starch, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, magnesium aluminum silicate, manesium silicate, magnesium trisilicate, montmorillonite, natto gum, potassium alginate, potassium carrageenan, propylene glycol alginate, sclerotium gum, sodium carboxymethyl dextran, sodium carrageenan, sodium polyacrylate starch, sodium silicoaluminate, starch/acrylates/acrylamide copolymer, tragacanth gum, xanthan gum, and mixtures thereof. The formulation can include gums in amounts generally ranging from about 0.05 to about 0.5 wt %.

The formulation can include polysaccharides. Exemplary polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, alkyl substituted celluloses, and mixtures thereof. The formulation can include polysaccharides in amounts generally ranging from about 5 to about 10 wt %.

The formulation can include pharmaceutical additives, such as those found in anti-fungal, anti-acne, anti-wrinkle, analgesics, and other pharmaceutically active topical applications. Exemplary pharmaceutical additives includekeratolytics, such as alpha-hydroxy acids, tocopherol sorbate, ascorbate, glycolic acid, salicylic acid, sulfur, lactic acid, pyruvic acid, resorcinol, and N-acetylcysteine; retinoids such as retinoic acid and its derivatives (e.g., and trans); antibiotics and antimicrobials such as benzoyl peroxide, octopirox, erythromycin, zinc, tetracyclin, triclosan, azelaic acid and its derivatives, phenoxy ethanol and phenoxy propanol, ethylacetate, clindamycin and meclocycline; sebostats such as flavinoids; alpha and beta hydroxy acids; and bile salts such as scymnol sulfate and its derivatives, deoxycholate, and cholate; non-steroidal anti-inflammatory drugs (NSAIDS), such as propionic acid derivatives, such as aspirin, acetaminophen, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen and bucloxic acid; acetic acid derivatives; fenamic acid derivatives; biphenvlcarboxylic acid. derivatives; and oxicams; steroidal anti-inflammatory drugs, such as hydrocortisone; salts of methddilizine and trimeprazine; salts of lidocaine, bupivacaine, chlorprocaine, di-bucaine, etidocaine, mepivacaine, tetracaine, dyclonine, hexylcaine, procaine, cocaine, ketamine, pramoxine, phenol, and mixtures thereof; pharmaceutically-acceptable salts of b-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, paromomycin, streptomycin, tobramycin, miconazole and amanfadine; tetracycline hydrochloride, erythromycin estolate, erythromycin stearate (salt), amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, parachlorometa xylenol, nystatin, tolnaftate, clotrimazole, and mixtures thereof. The formulation can include pharmaceutical additives in amounts generally ranging from about 0.1 to about 0.5 wt %.

The formulations of the present invention may further include a UV absorption agent, particularly in those embodiments wherein the composition is formulated as a sunblock, sunscreen or sunburn. UV absorption agents that may be suitable for use in the present invention include chemical and physical sunblocks. Non-limiting Representative examples of chemical sunblocks that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1-12), cinnamates (octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloy trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutyiphenol, and bis-ethylhexyloxyphenol methoxyphenyltriazine, 4-methylbenzylidenecamphor, and isopentyl 4-methoxycinnamate. Representative examples of physical sunblocks include kaolin, talc, petrolatum and metal oxides (e.g., titanium dioxide and zinc oxide).

The formulations of the present invention can include a structuring agent which are known to assist in providing rheological characteristics and contribute to stability. Representative examples of structuring agents that may be suitable for use in the present invention include waxes (e.g., natural waxes, such as candelilla wax, carnauba wax, Japan wax, esparto wax, cork wax, guaruma wax, rice germ oil wax, sugar cane wax, ouricury wax, montan wax, bees wax, shellac wax, spermaceti wax, lanolin (wool wax), preen gland fat, ceresin, ozokerite (mineral wax), petrolatum, paraffin waxes, microcrystalline waxes; chemically modified waxes (hard waxes), such as montan ester waxes, sasol waxes, hydrated jojoba waxes as well as synthetic waxes, such as polyalkylene waxes and polyethylene glycol waxes), and fatty acids, fatty alcohols and fatty acid esters (e.g., stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units).

Antioxidants that may be useful in the practice of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

Preservatives that may be suitable for use in the present invention include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, and thimerosal.

### Methods

Formulations containing AgNPs may provide protection against (e.g., inhibit or reduce) UV radiation-induced damage to skin, which may result from DNA damage, ROS production and apoptosis, inflammation and other long-term secondary changes e.g., recruitment of immune cells, altered stroma/extracellular matrix composition, and the like. The formulations of the present invention may also treat and ameliorate one or more symptoms associated with existing skin damage. The formulations may be used to treat non-cancerous skin conditions, such as those caused by UV-exposure, or alternatively, skin conditions resulting from cancer treatment, such as non-cancerous skin conditions resulting from exposure to radiation used in cancer treatment, or resulting from other undesired environmental exposure. Thus, the methods of the present invention and as used herein, the term “treat”, do not embrace treatment of existing skin cancer per se, but they do include application of the formulations in the context of radiation treatment or other types of cancer therapy that does cause damage to skin.

In some embodiments, the inventive method entails applying the formulation to the skin prior to exposure to UV radiation which includes both exposure to sun and exposure to synthetic radiation sources such as tanning beds.

In other embodiments, the method includes applying the formulation to skin that has been damaged. In some cases, the damage is caused by over-exposure to UV radiation, such as UVB radiation or other wavelengths of the UV spectrum. For example, these areas of the skin may be areas of sun burned or sun poisoned skin, or other forms of UV-induced damaged skin. Skin damage may also arise from other causes such as inflammation and natural ageing. Thus, broadly, the inventive formulations can be applied to skin wherein the cells are prone to mutation or in a mutated state, which may be induced by other means than UV radiation. Cells can be mutation prone when exposed to UV radiation, whereas a mutated state, for example, may be cells that are not normal or otherwise have some mutation in their DNA causing the cells to behave abnormally, such as uncontrollably reproduce, release proteins not released by normal cells or the like. Physically symptoms of such skin cells may include inflammation, or aging, e.g., wrinkled areas of skin.

The formulations can be applied directly by hand, or by using an applicator device, such as cotton swab, sponge, or the like. In yet other embodiments, the method may be practiced by applying the formulation to the skin by spraying using a spraying device, such as an aerosol device.

The AgNPs are applied to skin in an effective amount, which as used herein, refers to an amount that inhibits, reduces or otherwise mitigates the amount of skin damage that would be caused in the absence of the AgNPs, or in the case of treatment of damaged skin, an amount that reduces, ameliorates or otherwise mitigates at least one symptom of skin damage, such as inflammation. In general, an effective amount of AgNPs for these purposes ranges from about 0.002 to about 3 wt % based on the total weight of the formulation.

In some embodiments, the method of treating UV radiation-induced damage to skin cells can include examining genetic information regarding the skin cells of a subject prior to treatment with the formulation. For example, genetic information can include biomarkers present in the subject's skin cells. Exemplary biomarkers can include thymine dimer, CPD, 8-OHdG p53, p21/Cip1, and hydrogen peroxide (H₂O₂). These biomarkers may be indicative of the state of the skin cells, such as a mutated state as discussed herein.

The invention is now described in terms of the following, non-limiting examples.

### EXAMPLE 1

Synthesis and characterization of Silver Nanoparticles (AgNPs): AgNPs were synthesized utilizing published methods, such as those found in (Chen et al.,ACS Nano. 2010;4:6387-94; Lo'pez-Miranda et al., J Nanopart Res 2012;14:110-04), which is incorporated herein by reference, with some modifications. Such modifications may include optimizing the process parameters: Components ratios [Concentration of silver nitrate (1 mM), sodium borohydride (5 mM) and sodium citrate tribasic dehydrate (3 mM)], reaction sequence [sodium citrate tribasic dehydrate followed by silver nitrate and sodium borohydride], time of reaction [1 hour], and reaction temperature [37° C.]

In brief, about 100 μL of 0.1M AgNO₃ aqueous solution and about 100 μL of 0.3M trisodium citrate dehydrate aqueous solution were added to about 9.7 mL deionized water. About 100 μL of 0.5M sodium borohydride aqueous solution was mixed into the resulting solution through slow stirring. The formation of the AgNPs was confirmed when the solution changed from a transparent to a golden yellow color.

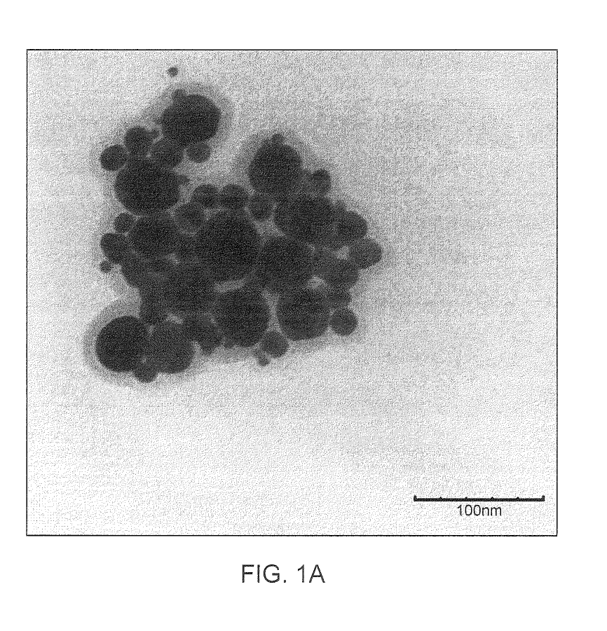

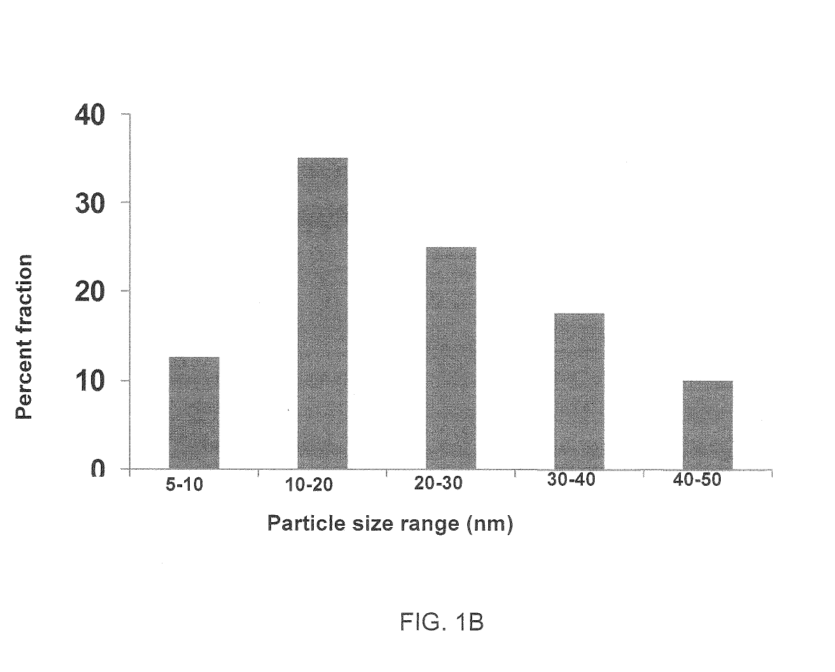

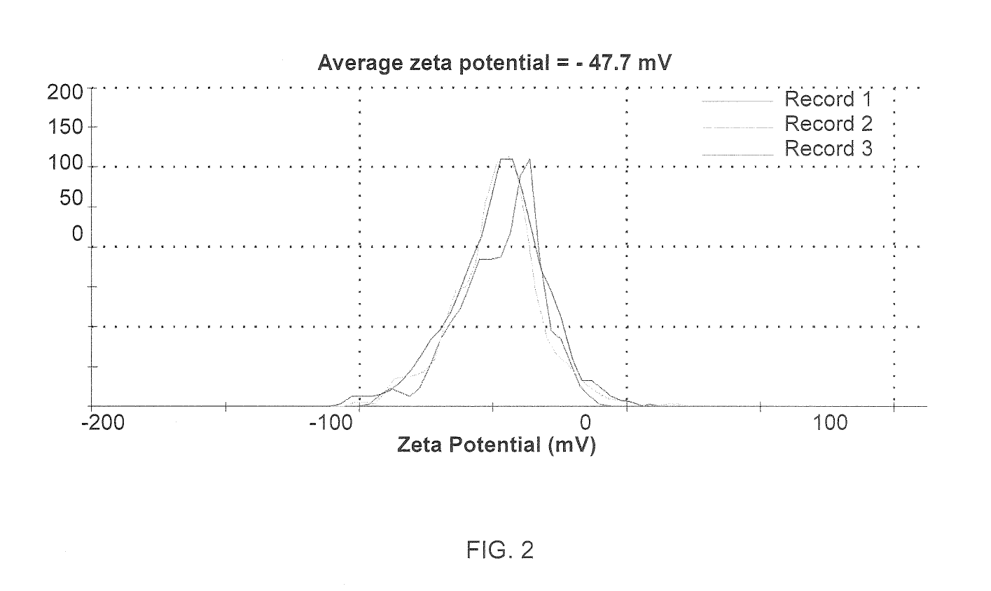

FIG. 1A depicts a transmission electron microscopy (TEM) micrograph of spherically shaped AgNPs. Particle size distribution as determined from the SEM micrograph is greater than about 75% of particles having a particle size ranging from about 10 nm to about 40 nm. AgNPs in a colloidal aqueous suspension are found to retain their stability, as determined by a high negative zeta potential (about −47.7 mV) as determined by Zeta sizer analysis as shown in FIG. 2. (Malvern Zetasizer (Malvern Instruments, Inc., Westborough, Mass.).

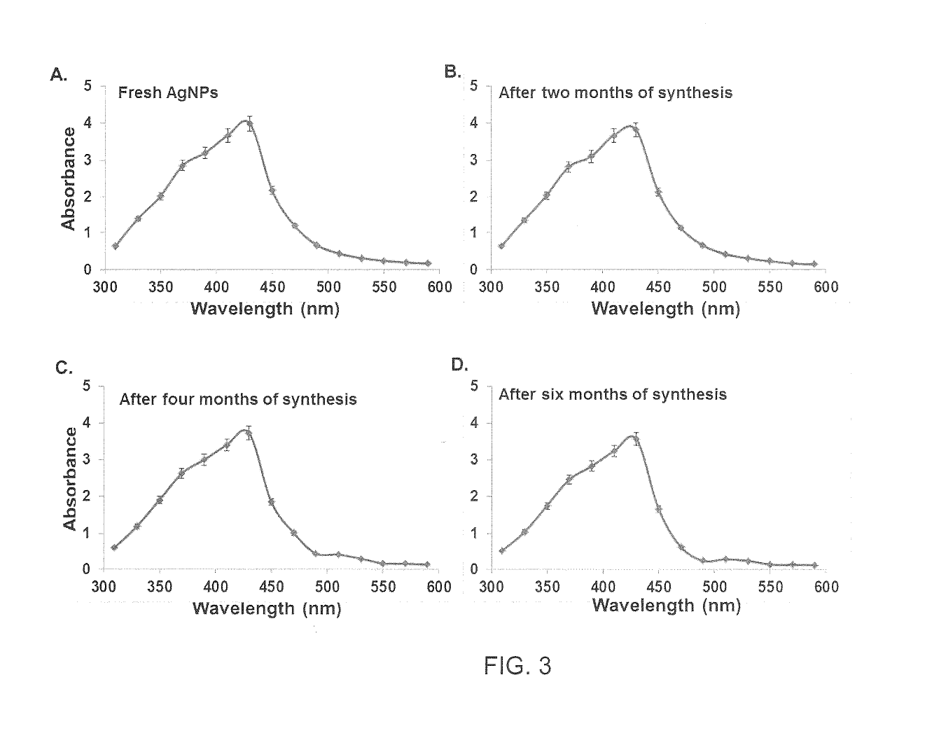

The AgNPs exhibit a characteristic surface plasmon peak at about 430 nm as determined by absorption spectroscopy. The absorption spectra depicted in FIGS. 3A-D demonstrate that the AgNPs were stable from immediately after synthesis (FIG. 3A) up to periods of 2 months (FIG. 3B), 4 months (FIG. 3C), and 6 months (FIG. 3D).

### EXAMPLE 2

### Human Keratinocytes (HaCaT) Cells Exposed to AgNPs

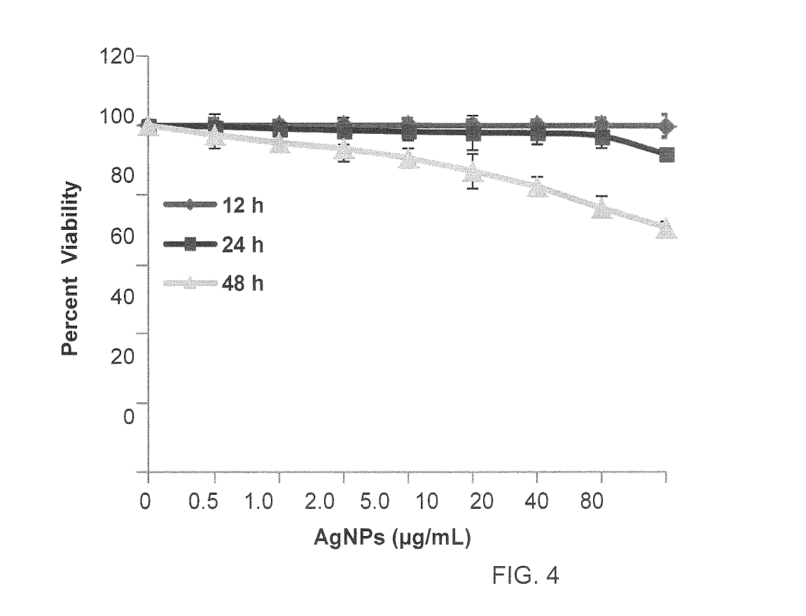

HaCaT cells were grown in 96 well plates (1×10⁴ cells/well) and treated with AgNPs having concentrations ranging from about 0.5 to about 80 μg/mL at about 70 to about 80% confluence. Percent viability of cells was measured by WST-1 assay (Roche Diagnostics, Mannheim, Germany) after 12, 24 and 48 h. The results of these measurements are depicted in FIG. 4. An optical density (OD) value of control cells was taken as 100% viability. More than 70% viability was observed even after 48 h of treatment at a maximum concentration (80 μg/mL) of AgNPs, suggesting minimal cytotoxicity of AgNPs. Data points in FIG. 4 are expressed as mean±SD; (n=3).

### EXAMPLE 3

### HaCaT Cells Pretreated with AgNPs

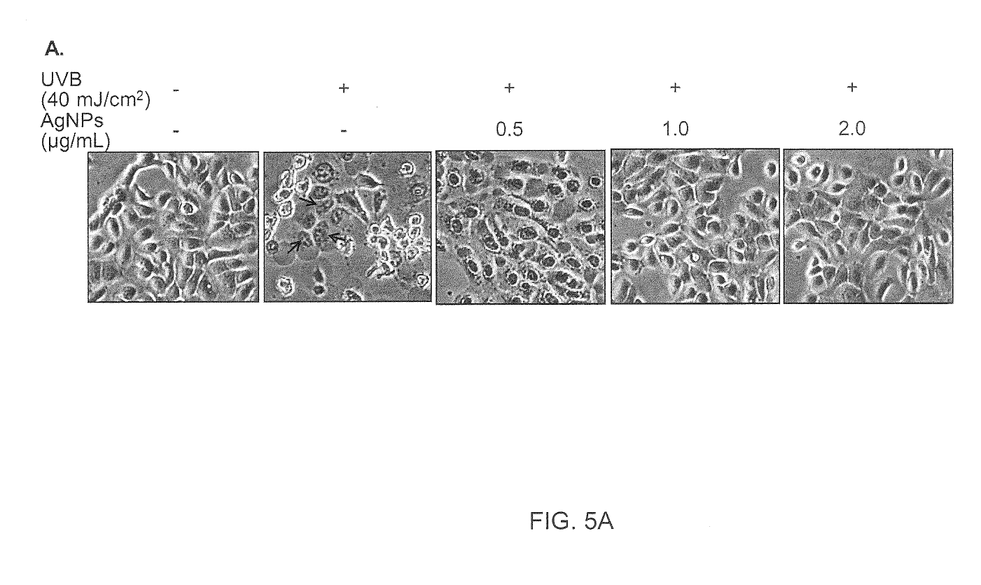

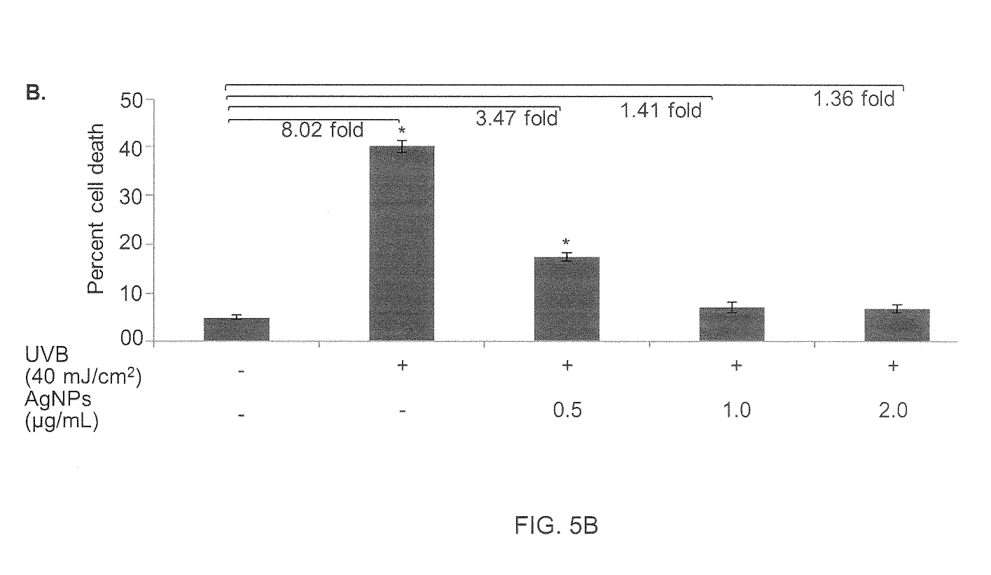

HaCaT cells were seeded in glass plates (1×10⁶ cells/plate) and allowed to attain 70-80% confluence. FIG. 5A depicts representative micrographs at ×200 magnification of five samples, which from left to right, are a control sample, which has not been treated with AgNPs or exposed to UV radiation; a UV sample, which has only been exposed to UV radiation (UVB, 40 mJ/cm²); a AgNPs(0.5 μg/mL)+UV sample, which has been pretreated with AgNPs for 3 hours and then exposed to UV radiation; a AgNPs(1.0 μg/mL)+UV sample; and a AgNPs(2.0 μg/mL)+UV sample. Following UV radiation exposure, the samples were examined after 24 h under a phase-contrast microscope. FIG. 5A depicts that after UVB-exposure, cells exhibited typical characteristic of apoptosis, such as cell shrinkage and nuclear fragmentation. However, pretreatment of cells with AgNPs retained their morphology after UVB-exposure. FIG. 5B depicts percent of surviving cells after treatment with AgNPs prior to UVB-exposure. These data demonstrate that pretreatment of keratinocytes with AgNPs suppressed UV-induced damage and apoptosis.

### EXAMPLE 4

### UVB Radiation-Induced Apoptosis in UVB-Exposed HaCaT

Four samples of HaCaT were prepared. These samples were: (1) Control, where the HaCaT were neither exposed to AgNPs nor to UV irradiation; (2) AgNPs, where the HaCaT were exposed to AgNPs (about 1 μg/mL) for 3 hours, but not to UV irradiation; (3) UV, where the HaCaT were exposed to UV irradiation (UVB radiation, 40 mJ/cm² in a range of 10 secons to 5 minutes in the absence of AgNP, and then harvested after 24 hours; and (4) AgNPs (1 μg/mL)+UV, where the HaCaT were exposed to AgNPs (about 1 μg/mL) for 3 hours, and then to UV irradiation (UVB radiation, 40 mJ/cm² in a range of 10 seconds to 5 minutes), and then harvested after 24 hours.

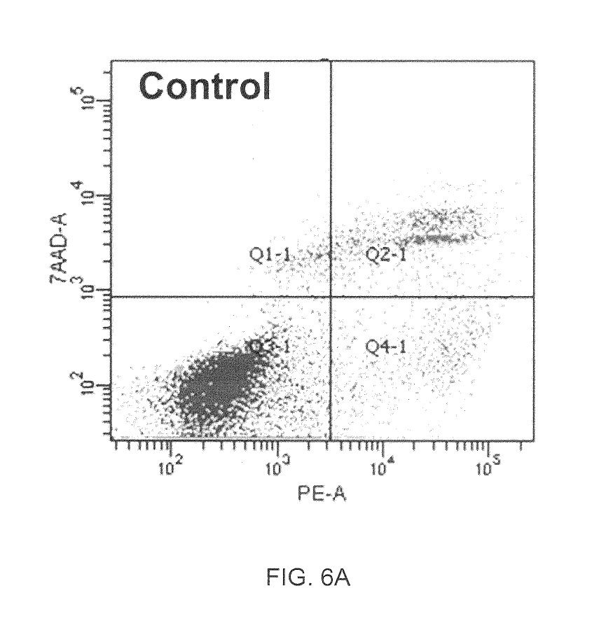

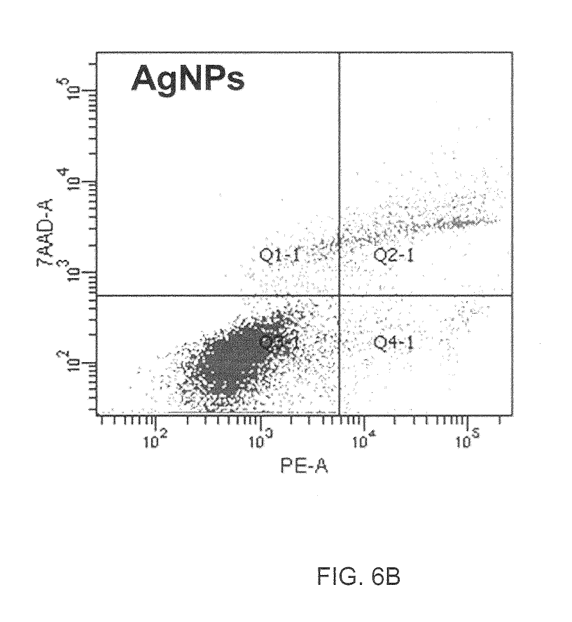

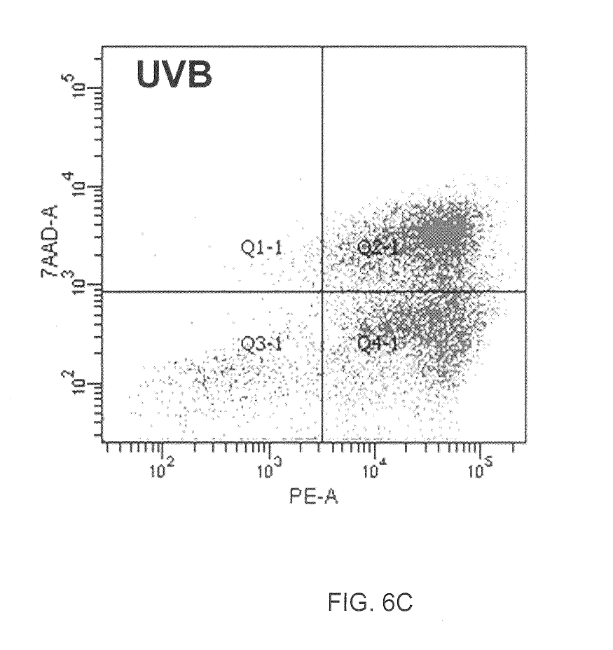

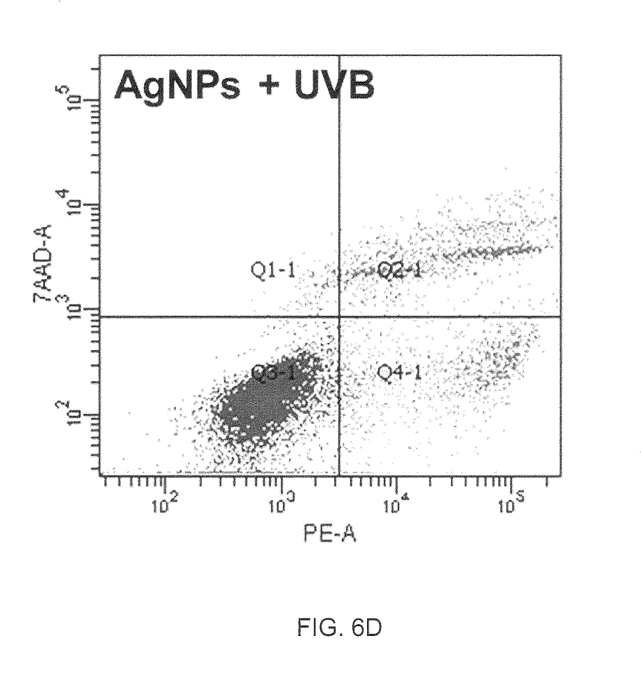

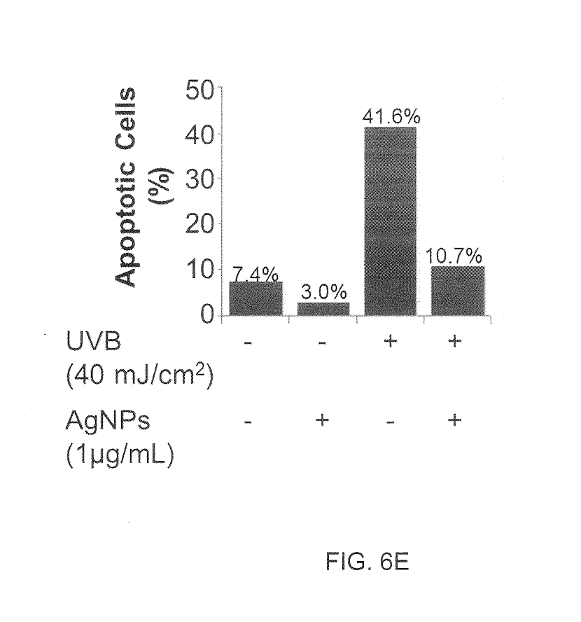

UVB-induced apoptosis in HaCaT was determined in the four samples by flow cytometry using an Annexin V. Apoptosis Detection Kit, available from BD Bioscience (San Diego, Calif.). After preparation of the samples, each sample was washed in phosphate buffered saline (PBS) and incubated with annexin-V-FITC and propidium iodide for cellular staining in binding buffer at room temperature for 10 min in the dark. The stained cells were analyzed by fluorescence activated cell sorting (FACS) using a FACS Caliber instrument (BD Biosciences, San Jose, Calif.) equipped with Cell Quest 3.3 software. The UV sample demonstrated significant induction of apoptosis (about 41.6%, P<0.005) compared to the Control sample (about 7.4%). The AgNPs+UV sample demonstrated reduction in apoptosis (about 10.7%, P<0.01) relative to the UV sample as depicted in FIG. 6A-E. The treatment of HaCaT with AgNPs significantly blocked UVB radiation-induced apoptosis.

### EXAMPLE 5

### Formation of Cyclobutane Pyrimidine Dimers (CPDs) in UVB-Exposed HaCaT Keratinocytes

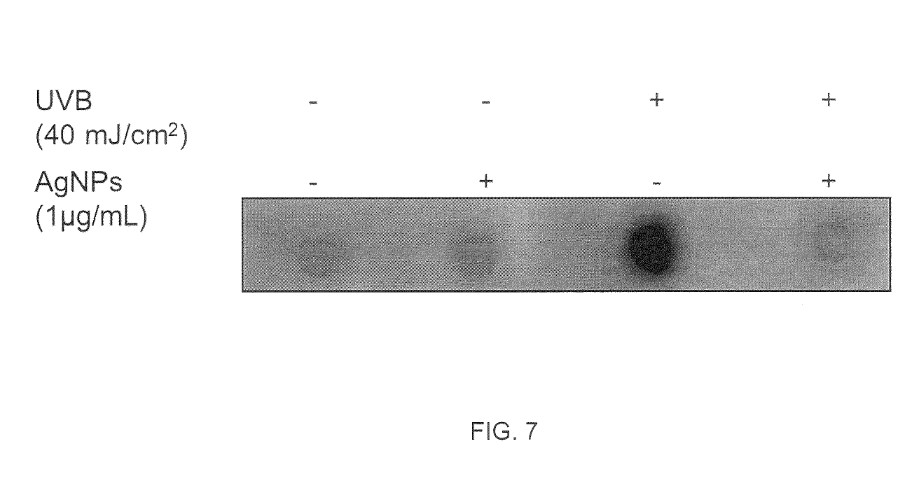

CPD formation was determined in the samples of Example 4 treating the samples with a CPD-specific antibody. FIG. 7 shows cytostained images of these samples after treatment with the antibody. CPD formation is indicted by the darker color in the dot-blot image of HaCaT cells. CPD-positive cells are not detectable in Control or AgNPs samples. However, CPD-positive cells are detected in UV and AgNPs+UV samples, as indicated by the darker color in the image. The number of CPD-positive cells was significantly lower in the AgNPs+UV samples indicating the ability of AgNPs to at least limit UV-induced damage in HaCaT, which can indicate protection from and/or repair of UV-induced damage in HaCaT.

### EXAMPLE 6

### G1/S Cell Cycle Arrest in UVB-Exposed HaCaT

In a UVB-exposed HaCaT, DNA damage response can culminate in activation of cell cycle checkpoints and appropriate DNA repair pathways. If the damage is irreversible, then apoptosis is initiated. The UVB-exposed HaCaT can make critical decisions about replication of DNA in G1 phase, and nucleotide injuries, such as CPDs, are repaired in G1 phase. A lack of fidelity in DNA replication and maintenance can result in deleterious mutations, leading to cell death or initiation of cancer. UVB exposure induces G2/M phase cell cycle arrest in HaCaT.

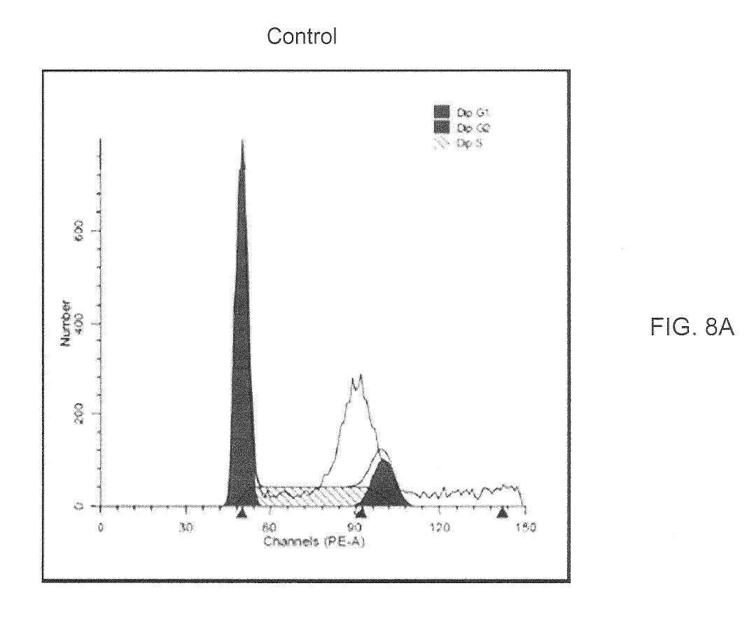

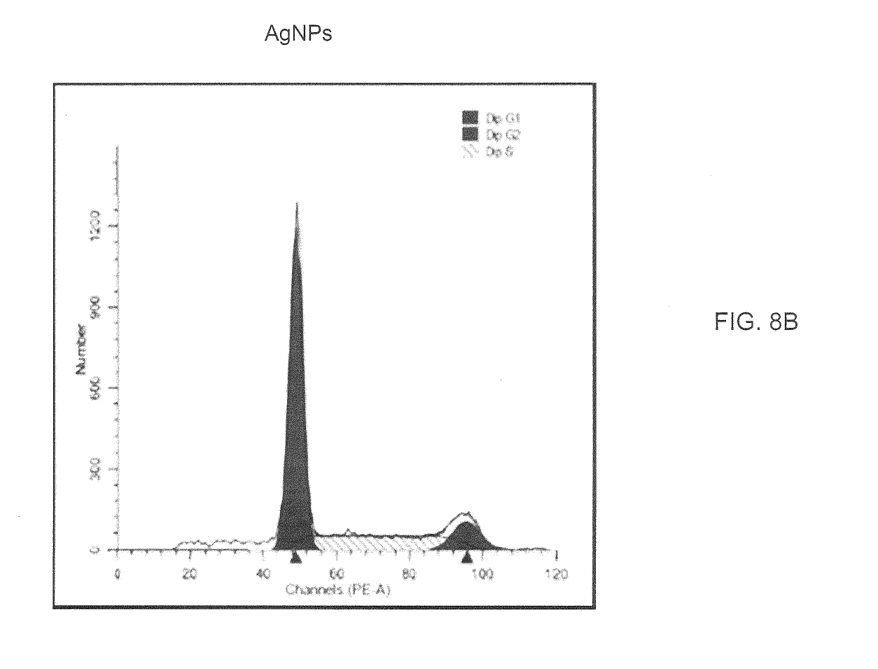

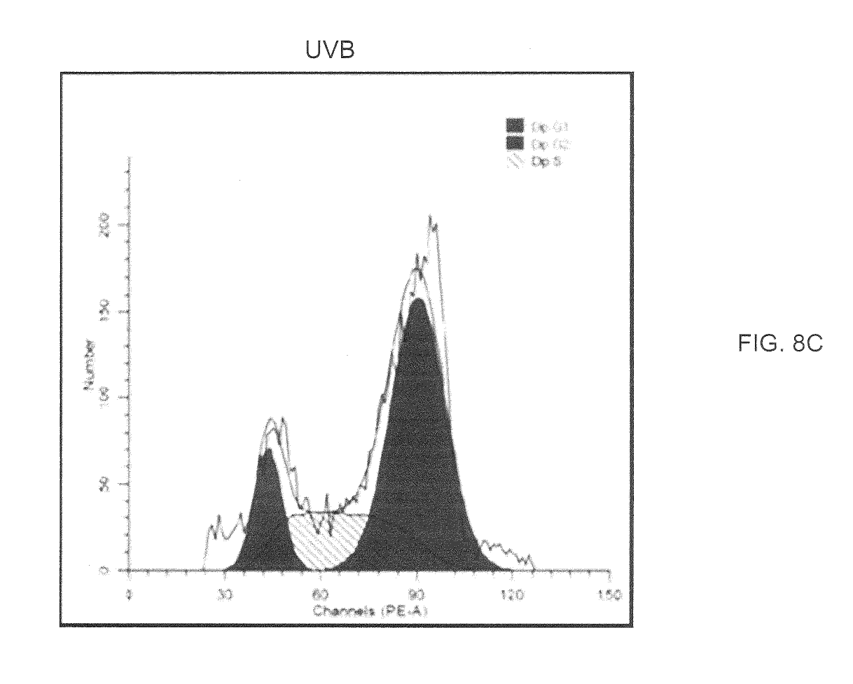

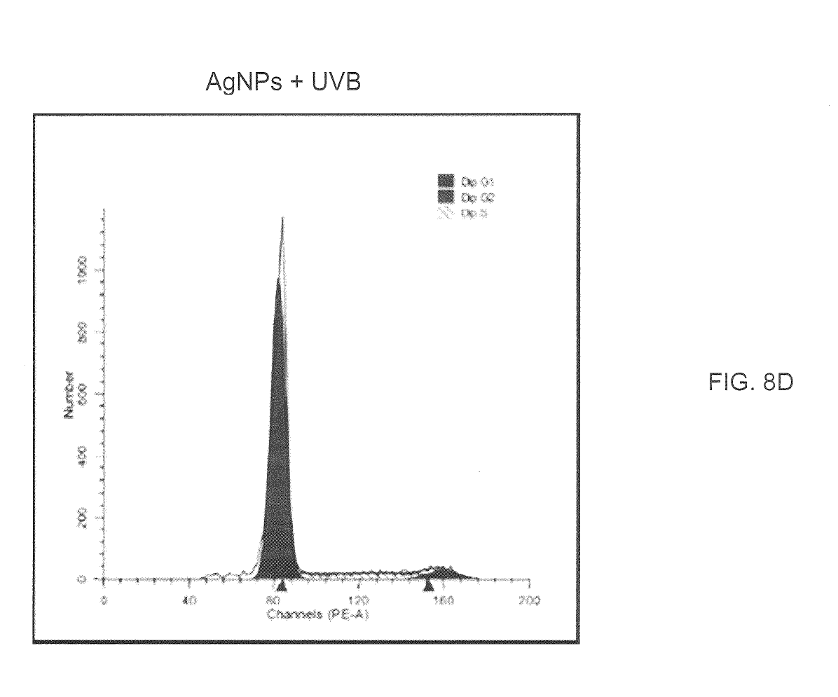

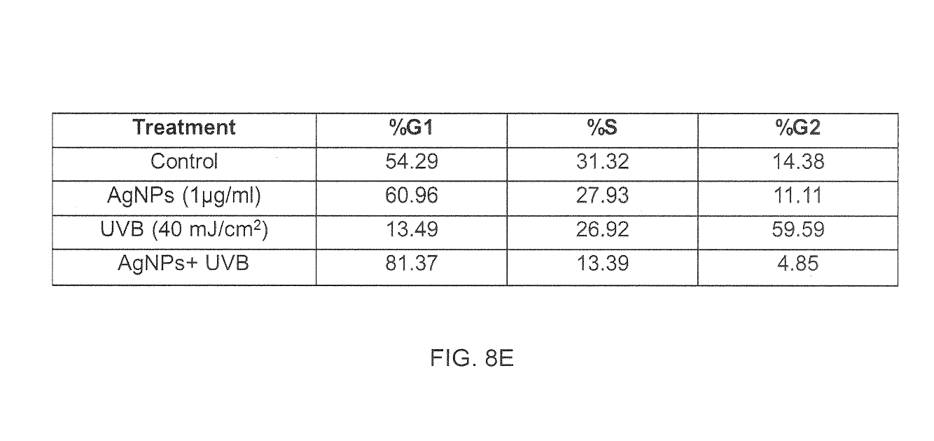

Cell cycle analysis was performed on the four samples of Example 4. In brief, cells (1×10⁶ cells/glass plate) were grown in complete medium for 24 h and then synchronized by culturing them in serum free medium for 48 h. Subsequently, cells were treated with AgNPs (1 μg/mL) in regular medium for 3 h prior to UVB-exposure. Following 24 h of UVB-exposure, cells were collected and fixed in 70% ethanol overnight at 4° C. Subsequently, cells were washed with PBS (0.1 M. pH-7.4), stained with propidium iodide and analyzed by flow-cytometry. Percentage of cell population in various phases of cell cycle was calculated using Mod Fit LT software. As shown in the results depicted in FIG. 8A-E, the UV sample demonstrated significant accumulation of HaCaT in the G2/M phase of the cell cycle. In contrast, the AgNPs+UV sample demonstrates a shift of cell accumulation in the G1/S phase. It is reported that during G1 phase, nucleotide-excision repair (NER) remove bulky lesions (CPDs) (LA et al., Photochem Photobiol. 1996;63:492-497; Branzei et al., Nat Rev Mol Cell Biol. 2008;9:297-308). Also, oxidation of guanine generates (oxoG), which is highly mutagenic can be removed by the base-excision repair (BER) which is active in G1 phase of the cell cycle. Therefore, the presence of AgNPs in the AgNPs+UV sample may facilitate DNA repair prior to cell replication. Thus, a significant reduction in mutated cellsmay be possible.

### EXAMPLE 7

### Cell-Cycle and Cell Survival Proteins in UV-Exposed HaCaT

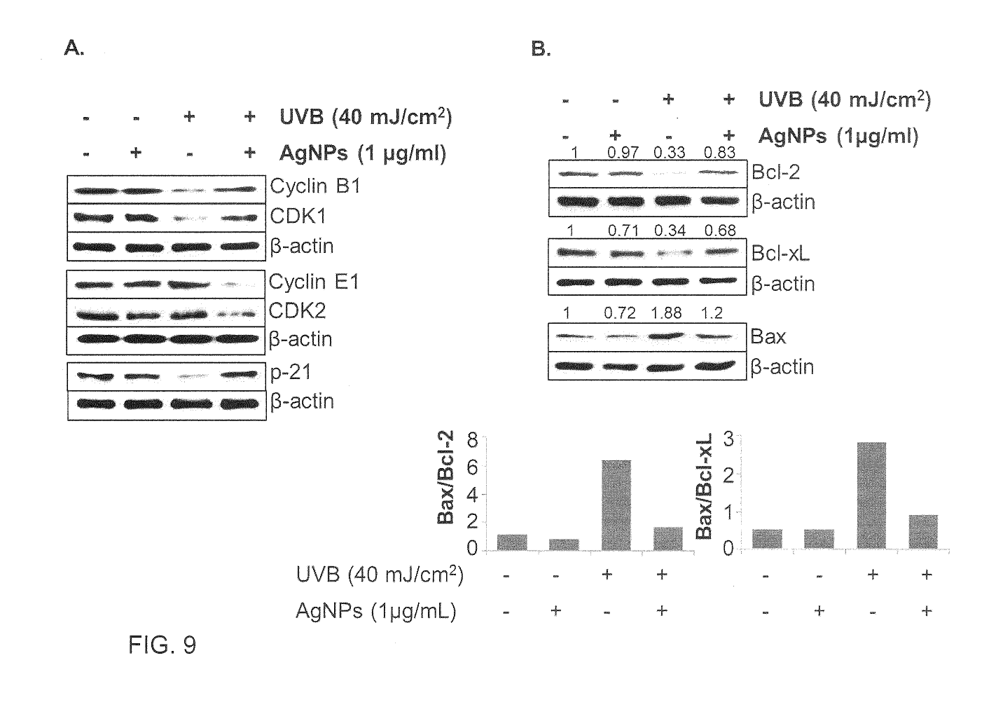

The four samples of Example 4 were evaluated for cell-cycle and cell survival protein content using antibody-based immuno detection. Protein content for cell cycle-associated proteins, Cyclin B1, Cyclin E1, CDK1, CDK2 and p21, was determined for each sample as depicted in FIG. 9A. β-actin was used as a loading control. The data show a decrease in the expression of Cyclin E1 and CDK2 in AgNPs+UV sample compared to other samples, whereas expression of Cyclin B1, CDK1 and p21 is restored to pre UV exposure level in AgNPs+UV sample.

Protein content for cell survival related proteins, Bcl-xL, Bcl-2 and Bax, was determined for each sample as depicted in FIG. 9B. β-actin was used as a loading control. Normalized densitometric values are indicated at the top of the bands. Bar diagram summarizing the effects of pretreatment with AgNPs prior to UVB-exposure on Bax/Bcl-2 ratio and Bax/Bcl-xL ratio. The data demonstrate that UVB-exposure resulted in downregulation of anti-apoptotic proteins, Bcl-2 and Bcl-xL, while the expression of pro-apoptotic protein Bax was increased. Expression of these proteins in HaCaT cells treated with AgNPs prior to UVB-exposure was relatively similar to that in the Control or AgNP samples, thus preventing apoptosis by maintaining the ratio of Bax/Bcl-2 and Bax/Bcl-xL.

### EXAMPLE 8

### Nucleotide Excision Repair (NER) Genes in AgNPs Pretreated UVB Exposed HaCaT

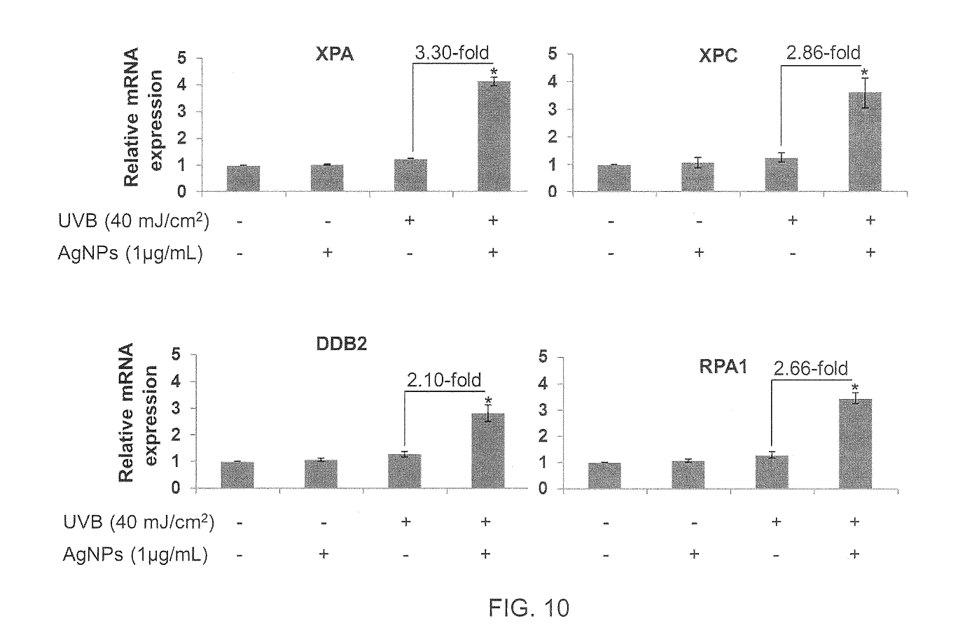

RNA isolated and mRNA expression of some NER genes (XPA, XPC, DDB2 and RPA1) was examined using real-time PCR for the four samples of Example 4. Data for each NER gene are depicted in FIG. 10. Bars represent the average of triplicates±S.D.; “*” (p<0.05) indicates statistical difference between AgNPs treated cells prior to UVB-exposure and cells exposed to UVB alone. As depicted in FIG. 10, the content of NER genes was significantly higher in a sample of AgNPs pretreated UV-exposed HaCaT cells.

### EXAMPLE 9

### Reactive Ion Species (ROS) Generation in UVB-Exposed HaCaT

In situ ROS generation was determined in the four samples of Example 4 by using a cell permeable dye, 2′, 7′-dichlorofluorescin diacetate (DCFH-DA). In operation, DCFH-DA is converted into a non-fluorescent reduced form (DCFH) after cleavage by cellular esterases present in HaCaT, and then DCFH is oxidized by ROS, present in HaCaT, into a fluorescent form (DCF).

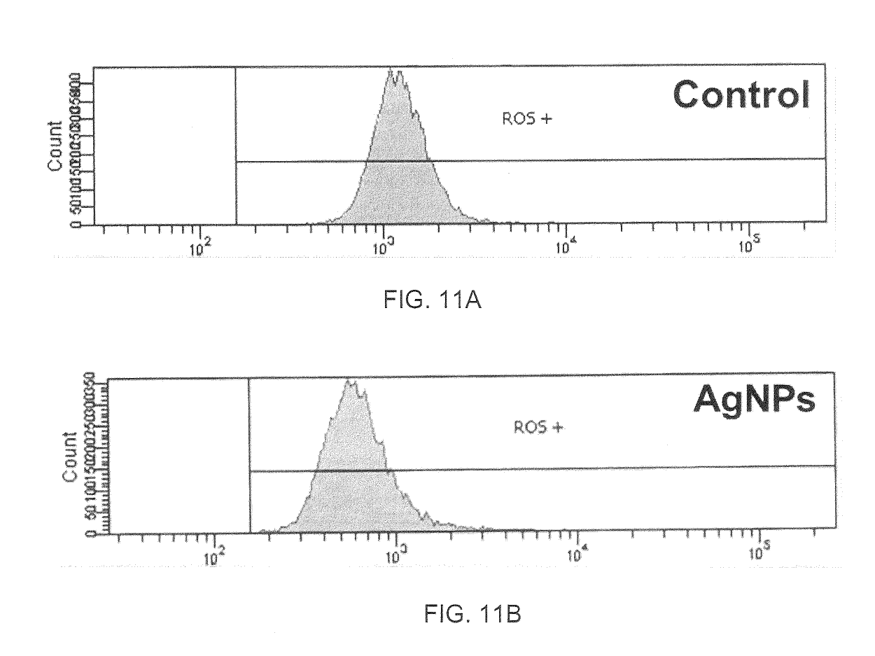

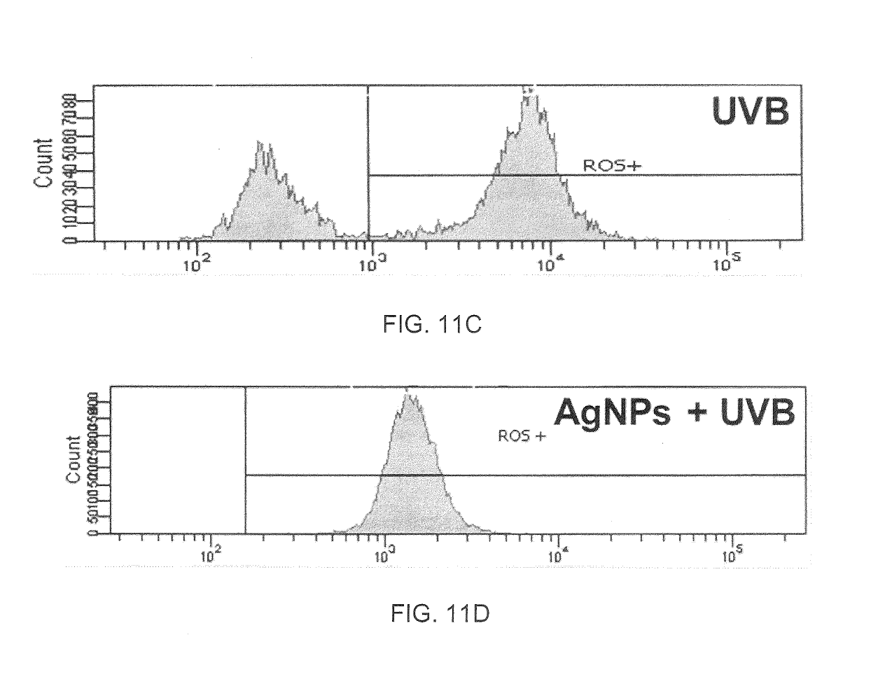

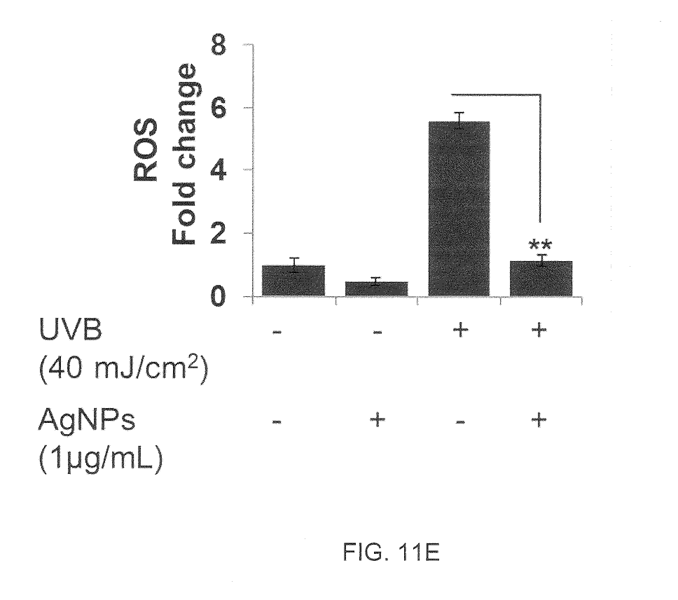

Each sample of Example 4 was incubated with DCFH-DA (Sigma Aldrich) in regular medium for about 30 min at about 37° C. treatment. After incubation, DCFH-DA-containing medium was removed, the samples were washed about 3 times with PBS, and then suspended in PBS. Fluorescence emission was measured at an emission wavelength of 530 nm using an excitation wavelength of about 485 nm by flow cytometry on a BD-FACS Canto Li (Becton-Dickson, San Jose, Calif.). The presence of AgNP (AgNPs+UV sample) facilitated about a 5 fold reduction in ROS generation compared to the UV sample as depicted in FIG. 11A-E.

### EXAMPLE 10

### Formation of CPDs in AgNPs Pretreated UVB-Exposed HaCaT Cells

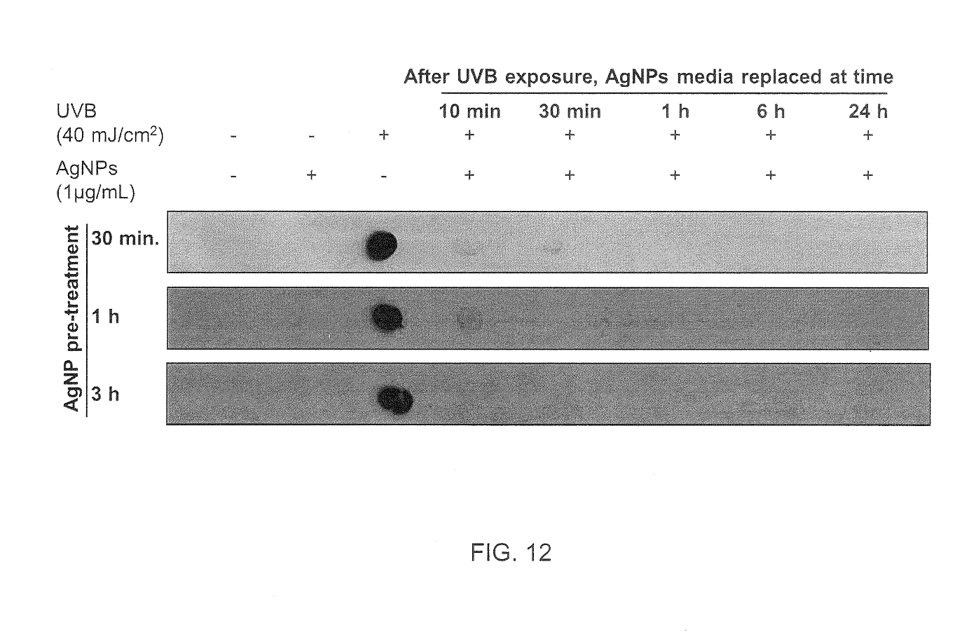

HaCaT cells were seeded in UV transparent petri dishes to reach 60-70% confluence and then treated with AgNPs for varying time intervals (30 minutes, 1 hour and 3 hours) before UVB irradiation (40 mJ/cm²). Media containing AgNPs was replaced with fresh media without AgNPs at different time periods (10 minutes, 30 minutes, 1 h, 6 h and 24 h). Genomic DNA from treated cells was collected after 24 hours of UVB-exposure and subjected to dot-blot analysis (FIG. 12) using an antibody specific to CPDs. The data demonstrate that AgNPs pretreatment reduced and/or repaired UVB-induced CPD formation in HaCaT cells even in case of 30 minutes pre-treatment and 10 minutes of incubation after UVB-exposure.

### EXAMPLE 11

### Formation of CPDs in AgNPs Post Treatmented UVB-Exposed HaCaT

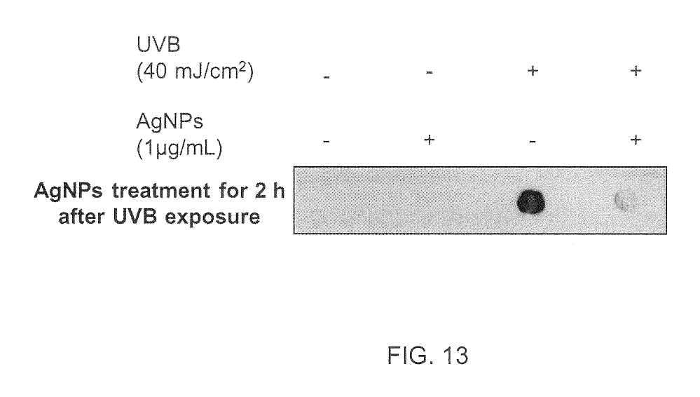

HaCaT cells were seeded in UV transparent petri dishes to reach 60-70% confluence and then exposed to UVB irradiation (40 mJ/cm²). After five minutes, cells were treated for 2 hours with AgNPs (1 μg/mL). Genomic DNA from treated cells was collected after 24 hours of UVB-exposure and subjected to dot-blot analysis (FIG. 13) using an antibody specific to CPDs. The data demonstrate that AgNPs post-treatment effectively repaired UVB exposure-induced DNA damage.

All publications cited in the specification, including patent publications and non-patent publications, are indicative of the level of skill of those skilled in the art to which this invention pertains. All these publications are herein incorporated by reference to the same extent as if each individual publication were specifically and individually indicated as being incorporated by reference.

Although the invention described herein has been described with reference to particular embodiments, it is to be understood that these embodiments are merely illustrative of the principle and applications described herein. It is therefore to be understood that numerous modifications may be made to the illustrative embodiments and that other arrangements may be devised without departing from the spirit and scope of the various embodiments described herein as defined by the amended claims.

We claim:

1. A method for reducing or inhibiting damage to skin, comprising:
applying to the skin prior to exposure to damaging source, a formulation containing an effective amount of silver nanoparticles (AgNPs).

2. The method of claim 1, wherein the damaging source includes ultraviolet (UV) radiation.

3. The method of claim 1, where the formulation includes a carrier.

4. The method of claim 3, wherein the formulation is in the form of an emulsion, a gel, an aerosol, or a non-aerosol.

5. The method of claim 3, wherein AgNPs range from about 0.002 to about 1 weight percent (wt %) based on the total weight of the formulation.

6. The method of claim 3, wherein the formulation is a sunscreen, and further comprises at least one UV absorbing agent.

7. The method of claim 3, wherein the formulation facilitates suppression of cyclobutane pyrimidine dimers (CPD) in DNA of skin cells relative to skin cells exposed to UV radiation in the absence of AgNPs.

8. The method of claim 3, wherein the formulation facilitates suppression of cell apoptosis in skin cells relative to skin cells exposed to UV radiation in the absence of AgNPs.

9. The method of claim 3, wherein the formulation facilitates arrest of G1 stage of cell cycle in skin cells relative to skin cells exposed to UV radiation in the absence of AgNPs.

10. The method of claim 3, wherein the formulation facilitates suppression of reactive oxidative species (ROS) in skin cells relative to skin cells exposed to UV radiation in the absence of AgNPs.

11. The method of claim 3, wherein the formulation facilitates increased expression of nucleotide excision repair (NER) genes in skin cells relative to skin cells exposed to UV radiation in the absence of AgNPs.

12. A method of treating skin damage, comprising:
applying to the damaged skin, a formulation containing an effective amount of silver nanoparticles (AgNPs).

13. The method of claim 12, wherein the skin damage is ultraviolet (UV) radiation-induced skin damage.

14. The method of claim 12, where the formulation includes a carrier.

15. The method of claim 14, wherein the formulation is in the form of an emulsion, a gel, an aerosol, or a non-aerosol.

16. The method of claim 12, wherein AgNPs range from about 0.002 to about 1 weight percent (wt %) based on the total weight of the formulation.

17. The method of claim 12, wherein prior to application of the formulation, further comprising:
determining a biomarker present in the UV radiation-damaged skin cells.

18. The method of claim 17, wherein the biomarker is selected from a group consisting of thymine dimer, cyclobutane pyrimidine dimers (CPD), 8-OHdG, p53, p21/Cip1, and hydrogen peroxide (H₂O₂).

19. The method of claim 12, wherein the AgNPS facilitate reduction of cyclobutane pyrimidine dimers (CPDs).

20. A topical formulation, comprising:
an effective amount of silver nanoparticles (AgNPs) to reduce or inhibit damage to skin, or to treat damaged skin; and
at least one dermatologically or cosmetically acceptable carrier.

21. The topical formulation of claim 20, wherein the at least one dermatologically or cosmetically acceptable ingredient include a carrier.

22. The topical formulation of claim 21, wherein the formulation is in the form of an emulsion, a gel, an aerosol, or a non-aerosol.

23. The topical formulation of claim 21, wherein AgNPs range from about 0.002 to about 1 weight percent (wt %) based on the total weight of the formulation.

24. The topical formulation of claim 20, wherein the topical formulation is a sunscreen, and further comprises at least one ultraviolet (UV) absorbing agent.
